(19) United States
(12) Patent Application Publication
(10) Pub. No.: US 2021/0401711 A1
(43) Pub. Date: Dec. 30, 2021

(54) COMPOSITION FOR STABILIZING POORLY SOLUBLE INGREDIENTS AND COSMETIC COMPOSITION COMPRISING SAME

(71) Applicant: LG HOUSEHOLD & HEALTH CARE LTD., Seoul (KR)

(72) Inventors: Soojung HA, Seoul (KR); Nam Seo SON, Seoul (KR)

(73) Assignee: LG Household & Health Care Ltd., Seoul (KR)

(21) Appl. No.: 17/294,829

(22) PCT Filed: Aug. 9, 2019
(86) PCT No.: PCT/KR2019/010157

§ 371 Date: May 18, 2021

(30) Foreign Application Priority Data
Nov. 19, 2018 (KR) 10-2018-0142721

(51) Int. Cl. A61K 8/55; A61K 8/44

(52) U.S. Cl. CPC A61K 8/553; A61K 2800/5424; A61K 2800/52; A61K 8/442

(57) ABSTRACT

The present invention relates to a composition for stabilizing poorly soluble ingredients, comprising hydrogenated lecithin and an anionic surfactant as effective components. In addition, the present invention relates to a method for stabilizing poorly soluble ingredients in cosmetic formulations by using said composition for stabilizing poorly soluble ingredients, a cosmetic composition comprising said composition for stabilizing poorly soluble ingredients and a poorly soluble component, and a preparation method of said cosmetic composition. The composition for stabilizing poorly soluble ingredients of the present invention stabilizes the poorly soluble ingredients in cosmetic formulations, thereby preventing same from being separated or precipitated. Accordingly, by overcoming the limitations of poorly soluble ingredients that tend to precipitate over time and thus were difficult to apply to cosmetic compositions, the present invention provides a cosmetic composition containing such poorly soluble ingredients and having various types of formulations.

### TECHNICAL FIELD

The present invention relates to a composition for stabilizing a poorly soluble ingredient, the composition containing hydrogenated lecithin and an anionic surfactant as active ingredients.

The present invention relates to a method for stabilizing a poorly soluble ingredient in a cosmetic formulation by using the composition for stabilizing a poorly soluble ingredient, to a cosmetic composition containing the composition for stabilizing a poorly soluble ingredient and a poorly soluble ingredient, and to a method for preparing the cosmetic composition.

### BACKGROUND ART

Among the ingredients that impart skin improving efficacy, such as whitening or wrinkle relief, to the skin in the development of cosmetics, there are many poorly soluble substances that dissolve in neither water nor oil. To impart skin improvement efficacy by containing such poorly soluble substances in cosmetics, a variety of methods have been attempted to stabilize the poorly soluble substances.

For example, a cream formulation employing a large amount of a polyol and an emulsifier having more packing ability, without containing water, was developed to stabilize ingredients that are easily precipitated in water, thereby allowing the cosmetic product to have spreadability and occlusiveness (Korean Patent Publication No. 10-2016-0031850). However, this cream formulation had other problems that have not yet been resolved, for example, instability over time, a stuffy texture due to the use of a large amount of a polyol, and the like.

In addition to such a formulation technique, studies have been conducted to develop a pharmaceutically usable composition by preparing a poorly soluble actives in the form of a polymer micelle or a nanoparticle using an amphiphilic block copolymer to thereby stabilize the poorly soluble actives (Korean Patent Publication No. 10-2010-0084399). However, such a composition still has limits in stabilizing a poorly soluble substance to thereby apply the same to products since aggregation of copolymers occurs and precipitation over time still remains during the manufacture of cosmetic products.

### DISCLOSURE

### Technical Problem

The present inventors studied to develop a cosmetic composition that sufficiently exerts functional effects of a poorly soluble ingredient and causes no precipitation of the poorly soluble ingredient, and as a result, the present inventors verified that a poorly soluble substance can be sufficiently stabilized in a cosmetic formulation by using a combination of hydrogenated lecithin and an anionic surfactant, thereby completing the present invention.

### Technical Solution

An object of the present invention is to provide a composition for stabilizing a poorly soluble ingredient, the composition containing hydrogenated lecithin and an anionic surfactant as active ingredients.

Another aspect of the present invention is to provide a cosmetic composition containing the composition for stabilizing a poorly soluble ingredient and a poorly soluble ingredient.

Another object of the present invention is to provide a method for stabilizing a poorly soluble ingredient in a cosmetic formulation, the method including mixing hydrogenated lecithin and an anionic surfactant.

Another aspect of the present invention is to provide a method for preparing a cosmetic composition containing a poorly soluble ingredient dissolved therein.

### Advantageous Effects

The composition for stabilizing a poorly soluble effective component in the present invention stabilizes a poorly soluble ingredient in a cosmetic formulation to prevent the separation and precipitation of the poorly soluble ingredient. Therefore, a poorly soluble ingredient-containing cosmetic composition having various types of formulations is provided by overcoming the limitation of a poorly soluble ingredient, which has been difficult to apply to cosmetic compositions due to the precipitation problem over time.

### BRIEF DESCRIPTION OF DRAWINGS

FIG. 1 shows an image of the formulation turbidity depending on the weight ratio of hydrogenated lecithin and cetyl phosphate.

FIG. 2 shows images of stability over time of Example 10 and Comparative Example 1 as observed by microscope.

### DETAILED DESCRIPTION OF THE INVENTION

The present invention will be specifically described as follows. Each description and embodiment disclosed in this disclosure may also be applied to other descriptions and embodiments. That is, all combinations of various elements disclosed in this disclosure fall within the scope of the present disclosure. Further, the scope of the present disclosure is not limited by the specific description below.

An aspect of the present invention provides a composition for stabilizing a poorly soluble ingredient, the composition containing hydrogenated lecithin and an anionic surfactant as active ingredients.

A poorly soluble ingredient with low solubility is generally difficult to contain in both oil and aqueous phases only by using a stabilizer used in cosmetics. The poorly soluble ingredient exists in an unstable state in a cosmetic formulation, and this instability increases with the passage of time, resulting in recrystallization and causing precipitation.

The present invention has been made in order to solve these problems, and there was developed a composition for use in stably containing a poorly soluble ingredient in a cosmetic formulation, and there was prepared a low-viscosity transparent formulation which forms a stable structure, that is, a micelle, without high-pressure emulsion, by mixing of an anionic surfactant with hydrogenated lecithin. Furthermore, the present invention is characterized in that by stabilization of a poorly soluble ingredient in the stable structure, the separation and precipitation of the poorly soluble ingredient is prevented.

As used herein, the term “hydrogenated lecithin” refers to a hydrogenated product of lecithin, which serves to increase the stability of a structure. The lecithin is a representative naturally derived surfactant, and is a generic term to designate any group of yellowish-brown fatty substances generated in animal and plant tissues composed of phosphoric acid, choline, fatty acids, glycerol, glycolipids, triglycerides, and phospholipids. The lecithin of the present invention includes all of naturally derived lecithin extracted from animals and plants, such as egg yolk, soybean, and corn, and microorganisms, such as E. coli, and synthetic lecithin. Specific examples of lecithin may be natural phospholipids, such as phosphatidylcholine, phosphatidylethanolamine, phosphatidylinositol, phosphatidylserine, lysophosphatidylcholine, sphingomyelin, yolk lecithin, and soybean lecithin, or synthetic lecithin, such as dilauroylphosphatidylcholine, dimyristoylphosphatidylcholine, dipalmitoylphosphatidylcholine, distearoylphosphatidylcholine, dioleoylphosphatidylcholine, palmitoylphosphatidylcholine, and oleoylphosphatidylcholine, but are not limited thereto. Typically, naturally derived lecithin may contain phosphatidylcholine in a content of 23 wt % to 95 wt % or phosphatidylethanolamine in a content of 20 wt % or less, but is not limited thereto.

The hydrogenated lecithin of the present invention may contain hydrogenated phosphatidylcholine in a content of 10 wt % to 99 wt %, and specifically 50 wt % to 90 wt %, but is not limited thereto.

The use of hydrogenated lecithin alone as a stabilizer in a cosmetic formulation results in the formation of a structure in the form of a liposome. However, this structure has an open shape and itself has high flexibility, and thus the structure is easily deformed or broken, thereby having difficulty in shape maintenance. This makes it difficult to secure the stability of a poorly soluble substance and results in a great change in stability over time. In the present invention, such a problem is solved by using an anionic surfactant together with hydrogenated lecithin.

The hydrogenated lecithin may be contained in a content of 0.1 wt % to 20 wt %, specifically 0.3 wt % to 10 wt %, 0.5 wt % to 5 wt %, or 1 wt % to 5 wt %, relative to the total weight of the composition for stabilizing a poorly soluble ingredient, but is not limited thereto.

Less than 0.1 wt % of hydrogenated lecithin may not sufficiently dissolve a poorly soluble ingredient, causing the deposition and precipitation of the poorly soluble ingredient. More than 20 wt % of hydrogenated lecithin may result in the increase in particle size of micelles, causing instability of a formulation.

As used herein, the term “anionic surfactant” refers to a surfactant containing only an anionic functional group as an ionic or ionizable group. The anionic surfactant, when mixed with hydrogenated lecithin, forms a low-viscosity transparent formulation, and helps to form a stable structure together with lecithin by adjusting the packing parameter of hydrogenated lecithin. By stabilizing a poorly soluble ingredient through the structure thus prepared, the separation and precipitation of the poorly soluble ingredient are prevented.

In the present invention, any anionic surfactant commonly known in the art can be used without limitation. Specifically, the anionic functional group contained in the anionic surfactant may be PO₄³⁻, —CO₂⁻, —SO₃⁻, —OSO₃⁻, —HPO₃⁻, —PO₃²⁻, —HPO₂⁻, —PO₂²⁻, —PO⁻, or a combination thereof, specifically phosphate (PO₄³⁻); or a metal salt of a carboxylic acid, such as a sodium salt, potassium salt, ammonium salt, or magnesium salt thereof, specifically a sodium salt of a carboxylic acid; or a combination thereof, but is not limited thereto. Besides, any anionic surfactant can be used without limitation to the type thereof as long as it can be mixed with hydrogenated lecithin to form a stable structure.

Specifically, the anionic surfactant may be an amino acid-derived surfactant, an alkyl phosphate, an alkyl sulfate, an alkyl ether sulfate, an alkyl mono-glyceryl ether sulfate, an alkyl sulfonate, an alkylaryl sulfonate, an alkyl sulfosuccinate, an alkyl ether sulfosuccinate, an alkyl sulfosuccinate, an alkyl amidosulfosuccinate, an alkyl carboxylate, an alkyl amidoethercarboxylate, an alkyl succinate, a fatty acyl sarcosinate, a fatty acyl amino acid, a fatty acyl taurate, a fatty alkyl sulfoacetate, or a combination thereof, and specifically may be an alkyl phosphate, but is not limited thereto.

The amino acid-derived surfactant is derived from a carboxylate salt of an amino acid, and specifically may include sodium dilauramidoglutamide lysine having a structure of glutamic acid-lysine-glutamic acid, derived from lysine.

The alkyl phosphate may be cetyl phosphate, PPG-10 cetyl phosphate, PPG-5-ceteth-10 phosphate, oleth-3 phosphate, oleth-10 phosphate, ceteth-10 phosphate, ceteth-10 phosphate, dicetyl phosphate, stearyl phosphate, or a mixture thereof, and more specifically cetyl phosphate, but is not limited thereto.

Specifically, for the purposes of the present invention, the anionic surfactant may be cetyl phosphate, sodium dilauramidoglutamide lysine, or a mixture thereof, and such a material may be mixed with hydrogenated lecithin to prepare a composition for stabilizing a poorly soluble ingredient in a cosmetic composition.

In the present invention, the anionic surfactant may be contained in a content of 0.001 wt % to 4 wt %, specifically 0.01 wt % to 2 wt %, 0.2 wt % to 1.0 wt %, or 0.1 wt % to 0.5 wt %, relative to the total weight of the composition for stabilizing a poorly soluble ingredient, but is not limited thereto.

Less than 0.001 wt % of the anionic surfactant cannot control the packing parameter of hydrogenated lecithin, failing to provide the preparation of a transparent formulation. More than 4 wt % of the anionic surfactant may result in the precipitation of the anionic surfactant.

The stability of the composition for stabilizing a poorly soluble ingredient may depend on the mixing ratio of the hydrogenated lecithin and the anionic surfactant contained in the composition. The mixing ratio of the hydrogenated lecithin and the anionic surfactant to increase the stability of the composition to the maximum level may depend on the type of the anionic surfactant.

Specifically, in a case where the anionic surfactant is cetyl phosphate, the mixing ratio of the hydrogenated lecithin and the anionic surfactant may be 1:0.005-40, specifically 1:0.02-1, 1:0.05-1, 1:0.2-1, 1:0.2-0.5, 1:0.2-0.3, or 1:0.2-0.25, but is not limited thereto.

In a case where the anionic surfactant is sodium dilauramidoglutamide lysine, the mixing ratio of hydrogenated lecithin and the anionic surfactant may be 1:0.005-40 and, specifically 1:0.1-2, 1:0.3-2, greater than 1:0.6 and no greater than 1:2, greater than 1:0.6 and no greater than 1:1, greater than 1:0.6 and no greater than 1:0.8, or 1:0.8 to 1:1, but is not limited thereto.

The composition for stabilizing a poorly soluble ingredient of the present invention may further contain an oil. In a case where the composition for stabilizing a poorly soluble ingredient of the present invention further contains an oil, the effect of stabilizing the poorly soluble ingredient can be further improved, and as for the oil, any oil that is commonly used in the corresponding technical field can be used without limitation.

Specifically, the oil may be a hydrocarbon-based oil, an ester-based oil, a silicone oil, or a mixture thereof, but is not limited thereto. Examples of the hydrocarbon-based oil may include hydrogenated polyisobutene, hydrogenated polydecene, paraffin, or a combination thereof; examples of the ester-based oil may include dipentaerythrityl hexa C₅₋₉ acid ester, diisostearyl maleate, C₁₂₋₁₅ alkyloctanoate, myristylactate, cetylethinehexanoate, cetyloctanoate, isopropyl myristate, octyldodecyl myristate, hexyllaurate, pentaerythrityl tetraethylhexanoate, diglyceryl triisostearate, or a combination thereof; examples of the silicone oil may include dimethicone, cyclomethicone, polydimethylsiloxane, methylphenyl polysiloxane, methylcyclopolysiloxane, octamethyl cyclotetrasiloxane, decamethyl cyclopentasiloxane, dodecamethyl cyclohexasiloxane, tetradecamethyl hexasiloxane, octamethyl trisiloxane, or a combination thereof. For the purpose of the present invention, hydrogenated polyisobutene may be used, but the oil is not limited thereto.

An exemplary embodiment of the present invention confirmed that when a composition for stabilizing a poorly soluble ingredient was prepared by containing hydrogenated polyisobutene together with hydrogenated lecithin and cetyl phosphate and the composition was contained together with a poorly soluble ingredient in a cosmetic formulation, the appearance of the cosmetic formulation was maintained and precipitation did not occur, even with the passage of 4 weeks, indicating that the poorly soluble ingredient was stably dissolved for a long period of time (Table 7).

Another aspect of the present invention is to provide a cosmetic composition containing the composition for stabilizing a poorly soluble ingredient and a poorly soluble ingredient.

The “composition for stabilizing a poorly soluble ingredient” is as described above.

In the present invention, the “poorly soluble ingredient” refers to a substance that is sufficiently dissolved in neither water nor oil and thus exists in an unstable state, and can be used in a cosmetic composition to impart helpful efficacy to the skin, such as wrinkle relief, skin whitening, or skin moisturizing.

Specifically, the poorly soluble ingredient contained in the composition of the present invention may be at least one type selected from the group consisting of cedrol, formononetin, magonolol, honokiol, phloretin, ceramide, a titrated extract of Centella asiatica, fisetin, daidzein, genistein, glycitein, adenosine, polydatin, retinol, γ-aminobutyric acid, arbutin, macelignan, acetyl phytosphingosine, hydroquinone, hydroxyanisole, ascorbic acid, kojic acid, and retinoids, and specifically cedrol, but is not limited thereto. Any ingredient can be applied to the present invention without limitation to the type thereof as long as the ingredient is used to provide beneficial efficacy to the skin.

An exemplary embodiment of the present invention confirmed that the precipitation of cedrol could be prevented by containing cedrol together with a mixture of hydrogenated lecithin and cetyl phosphate in a cosmetic formulation. As such, the cosmetic composition of the present invention has effects of preventing the separation and precipitation of a poorly soluble ingredient by stably containing the poorly soluble ingredient, and specifically has an advantage of causing no separation and precipitation under a temperature condition of −20° C. to 60° C. That is, the cosmetic composition of the present invention can maintain a stable form thereof under conditions of low and high temperatures as well as room temperature for a long period of time.

The composition for stabilizing a poorly soluble ingredient of the present invention can stabilize a poorly soluble ingredient in a variety of formulations including from high-viscosity formulations to low-viscosity formulations. The cosmetic composition containing a poorly soluble ingredient of the present invention can be formulated into any formulation that is commonly prepared in the art, and has various applications to formulations, such as high-viscosity creams, low-viscosity creams, lotions, essences, mists, and sprays.

For example, the cosmetic composition may be prepared into a formulation selected from the group consisting of skins, lotions, essences, creams or eye creams, solutions, externally applied ointments, foams, nutritious skin lotions, softening skin lotions, masks, packs, softeners, milky lotions, makeup bases, soaps, liquid cleaning agents, bath preparations, sun screen creams, sun oils, suspensions, emulsions, pastes, gels, powders, surfactant-containing cleansing agents, oils, powder foundations, emulsion foundations, wax foundations, patches, and sprays, but is not limited thereto.

In addition, the cosmetic composition of the present invention may further contain at least one type of cosmetically acceptable carrier that is mixed in typical skin cosmetic materials, and typical ingredients, such as oils, water, surfactants, moisturizers, lower alcohols, thickeners, chelating agents, pigments, preservatives, and fragrances, may be appropriately mixed, but are not limited thereto. The cosmetically acceptable carrier contained in the cosmetic composition of the present invention may vary depending on the formulation.

Another object of the present invention is to provide a method for stabilizing a poorly soluble ingredient in a cosmetic formulation, the method including mixing hydrogenated lecithin and an anionic surfactant.

The terms “hydrogenated lecithin”, “anionic surfactant”, and “poorly soluble ingredient” are as described above.

Specifically, the composition obtained by mixing hydrogenated lecithin and an anionic surfactant is mixed with a poorly soluble ingredient, so that the poorly soluble ingredient can be stabilized in the cosmetic composition.

The hydrogenated lecithin and the anionic surfactant may be mixed by sequential, reverse, or simultaneous addition, and the poorly soluble ingredient may be added after both the hydrogenated lecithin and anionic surfactant are mixed, or the poorly soluble ingredient may be first mixed with the hydrogenated lecithin or anionic surfactant before mixing, and then the other ingredient may be added and mixed. The timing and order of adding and mixing these are not limited as long as the poorly soluble ingredient can be stabilized after the cosmetic composition containing all of hydrogenated lecithin, the anionic surfactant, and the poorly soluble ingredient is finally prepared.

The anionic surfactant may be cetyl phosphate, sodium dilauramidoglutamide lysine, or a mixture thereof.

Another aspect of the present invention is to provide a method for preparing a cosmetic composition containing a poorly soluble ingredient dissolved therein, the method including: (S1) adding to a solvent a poorly soluble ingredient, hydrogenated lecithin, and an anionic surfactant; and (S2) adding the resultant product in step (S1) to an aqueous phase.

The anionic surfactant may be cetyl phosphate, sodium dilauramidoglutamide lysine, or a mixture thereof.

The terms “hydrogenated lecithin”, “anionic surfactant”, and “poorly soluble ingredient” are as described above.

The cosmetic composition obtained by the preparation method of the present invention stably contains a poorly soluble ingredient, so that the cosmetic composition can be stably maintained without the separation and precipitation of the poorly soluble ingredient under various formulation and temperature conditions for a long period of time.

### EXAMPLES

Hereinafter, the present invention will be described in more detail through the following exemplary embodiments. However, the exemplary embodiments according to the present invention may be modified in many different forms, and the scope of the present invention shall not be construed as being limited to the exemplary embodiments described below. The exemplary embodiments of the present invention are provided to illustrate the present invention more completely to those skilled in the art.

### Experimental Example 1: Verification of Formulation Appearance and Turbidity of Compositions for Stabilizing Poorly Soluble Ingredient

1-1. Preparation of Compositions for Stabilizing Poorly Soluble Ingredient

As shown in Table 1 below, cosmetic compositions containing various types and combinations of ingredients were prepared.

First, an oily phase and an aqueous phase were separately dissolved by heating at 65° C. The oily phase was slowly added while the aqueous phase was stirred using a homomixer at 2000 rpm. After the oily phase was completely added, stirring was conducted using the homomixer at 2000-2500 rpm for 3 minutes, followed by cooling to 28° C. to thereby prepare the cosmetic compositions. The corresponding compositions for stabilizing a poorly soluble ingredient were prepared as skin formulations.

TABLE 1

| Classification | Raw material | Example 1 | Example 2 | Example 3 | Example 4 | Example 5 |
|---|---|---|---|---|---|---|
| Oily phase | Ethanol | 2 | 2 | 2 | 2 | 2 |
| | Dipropylene | 1 | 1 | 1 | 1 | 1 |
| | glycol | | | | | |
| | Hydrogenated | 1 | 1 | 1 | 1 | 1 |
| | lecithin | | | | | |
| | Cetyl phosphate | 0.05 | 0.1 | 0.15 | 0.2 | 0.25 |
| | Fragrance | 0.1 | 0.1 | 0.1 | 0.1 | 0.1 |
| Aqueous | Purified water | Remainder | Remainder | Remainder | Remainder | Remainder |
| phase | Arginine | 0.025 | 0.05 | 0.08 | 0.1 | 0.13 |
| | Glycerin | 8 | 8 | 8 | 8 | 8 |
| | Propanediol | 5 | 5 | 5 | 5 | 5 |
| | 1,2-Hexanediol | 1.5 | 1.5 | 1.5 | 1.5. | 1.5 |
| | Panthenol | 1.5 | 1.5 | 1.5 | 1.5 | 1.5 |

TABLE 2

| Classi- | | | | |
|---|---|---|---|---|
| fication | Raw material | Example 6 | Example 7 | Example 8 |
| Oily phase | Ethanol | 2 | 2 | 2 |
| | Dipropylene glycol | 1 | 1 | 1 |
| | Hydrogenated | 1 | 1 | 1 |
| | lecithin | | | |
| | Sodium | 0.3 | 0.6 | 0.8 |
| | dilauramidoglutamide | | | |
| | lysine | | | |
| | Fragrance | 0.1 | 0.1 | 0.1 |
| Aqueous | Purified water | Up to 100 | Up to 100 | Up to 100 |
| phase | Glycerin | 8 | 8 | 8 |
| | Propanediol | 5 | 5 | 5 |
| | 1,2-Hexanediol | 1.5 | 1.5 | 1.5 |
| | Panthenol | 1.5 | 1.5 | 1.5 |

1-2. Measurement of Formulation Appearance and Turbidity Immediately after Preparation

To investigate whether the formulations of the compositions for stabilizing a poorly soluble ingredient prepared in Preparation Example 1 were transparent, the appearance of the compositions immediately after the preparation in Preparative Example 1 was observed. For comparison of formulation appearance, the extent of turbidity was checked with the naked eye, and the turbidity was measured using a turbidimeter. The turbidity was measured by using the model TL2350 (HACH), which measures the turbidity using a light scattering method, and the turbidity was expressed as a mean value of a total of five measurements. The measurement was conducted at 25° C. for each example. The results are shown in Table 3.

TABLE 3

| | Example 1 | Example 2 | Example 3 | Example 4 | Example 5 | Example 6 | Example 7 | Example 8 |
|---|---|---|---|---|---|---|---|---|
| Appearance | Suspended | Semi- | Semi- | Transparent | Transparent | Suspended | Transparent | Transparent |
| | | suspended | transparent | | | | | |
| Turbidity | 1052 | 305 | 52.77 | 35.60 | 12.10 | 1120 | 38 | 16.20 |
| (NTU) | | | | | | | | |

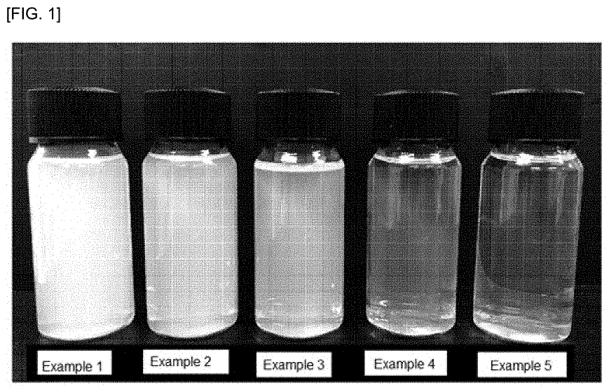

As can be seen from Table 3 and FIG. 1, the compositions became more transparent as the content of cetyl phosphate or sodium dilauramidoglutamide lysine compared with the content of hydrogenated lecithin increased. Both the naked-eye observation results and the turbidity measurement results were as described above.

1-3. Comparison of Formulation Appearance and Turbidity Over Time

To find the conditions of hydrogenated lecithin and an anionic surfactant which are suitable for stabilizing a poorly soluble ingredient, the formulation stability of the cosmetic compositions of Examples 1 to 8 over time was evaluated. A formulation appearance comparison experiment was performed by the same method as in Experimental Example 1, and the results are shown in Tables 4 and 5.

TABLE 4

| | Example 1 | Example 2 | Example 3 | Example 4 | Example 5 |
|---|---|---|---|---|---|
| Immediately after | Suspended | Semi- | Semi- | Transparent | Transparent |
| preparation | | suspended | transparent | | |
| Turbidity (NTU) | 1052 | 305 | 52.77 | 35.60 | 12.10 |
| Next day (room | Suspended | Suspended | Semi- | Transparent | Transparent |
| temperature) | | | transparent | | |
| Turbidity (NTU), room | 1232 | 380 | 60.22 | 36.12 | 12.68 |
| temperature | | | | | |
| Turbidity (NTU), −20° C. | 1488 | 450 | 112.20 | 45.21 | 16.74 |
| One week after | Suspended | Suspended | Suspended | Transparent | Transparent |
| preparation (room | | | | | |
| temperature) | | | | | |
| Turbidity (NTU), room | 1300 | 412 | 53.24 | 35.16 | 15.10 |
| temperature | | | | | |
| Turbidity (NTU), −20° C. | 1670 | 705 | 705 | 44.04 | 20.48 |
| Four weeks after | Suspended | Suspended | Suspended | Transparent | Transparent |
| preparation (room | | | | | |
| temperature) | | | | | |
| Turbidity (NTU), room | 1392 | 410 | 52.76 | 40.10 | 16.88 |
| temperature | | | | | |
| Turbidity (NTU), −20° C. | 1710 | 762 | 762 | 46.10 | 24.04 |

TABLE 5

| | Example 6 | Example 7 | Example 8 |
|---|---|---|---|
| Immediately after | Suspended | Transparent | Transparent |
| preparation | 1120 | 38 | 16.20 |
| Turbidity (NTU) | | | |
| Next day (room | Suspended | Semi-transparent | Transparent |
| temperature) | 1200 | 50.2 | 16.22 |
| Turbidity (NTU), room | | | |
| temperature | 1420 | 52 | 21.20 |
| Turbidity (NTU), −20° C. | | | |
| One week after | Suspended | Semi-transparent | Transparent |
| preparation (room | 1250 | 54 | 18.42 |
| temperature) | | | |
| Turbidity (NTU), room | | | |
| temperature | 1580 | 60.2 | 25.20 |
| Turbidity (NTU), −20° C. | | | |
| Four weeks after | Suspended | Semi-transparent | Transparent |
| preparation (room | 1340 | 54.6 | 18.80 |
| temperature) | | | |
| Turbidity (NTU), room | | | |
| temperature | 1740 | 67 | 27.32 |
| Turbidity (NTU), −20° C. | | | |

As shown in Table 4, as the content of cetyl phosphate relative to the content of hydrogenated lecithin increased, a transparent state was maintained without appearance change under the room temperature condition. However, Examples 1 to 3 with a relatively low content of cetyl phosphate showed a change in appearance over time and became turbid.

For comparison of long-term stability, Examples 1 to 3, employing 0.15 wt % or less of cetyl phosphate on the basis of 1 wt % of hydrogenated lecithin, showed a change in appearance and became turbid within one week from the next day after the preparation, and suspension also occurred with respect to the stability at −20° C.

Examples 4 and 5, employing 0.2 wt % or more of cetyl phosphate, maintained the appearance even four weeks after the preparation. These results indicate that the weight ratio of hydrogenated lecithin and cetyl phosphate is important, wherein the stability of the composition for stabilizing a poorly soluble ingredient is maintained only when the amount of cetyl phosphate is 0.2 parts by weight or more relative to 1 part by weight of hydrogenated lecithin.

As shown in Table 5, as the content of sodium dilauramidoglutamide lysine relative to the content of hydrogenated lecithin increased, a transparent state was maintained without appearance change under the room-temperature condition. However, Examples 6 to 7, with a relatively low content of sodium dilauramidoglutamide lysine, showed a change in appearance over time, and thus became turbid.

For comparison of long-term stability, Examples 6 and 7, employing 0.6 wt % or less of sodium dilauramidoglutamide lysine on the basis of 1 wt % of hydrogenated lecithin, showed a change in appearance and became turbid from the next day, and suspension also occurred with respect to the stability at −20° C.

Meanwhile, Example 8, employing more than 0.6 wt % of sodium dilauramidoglutamide lysine, maintained the appearance even four weeks after the preparation. These results indicate that the weight ratio of hydrogenated lecithin and sodium dilauramidoglutamide lysine is important, and the stability of the composition for stabilizing a poorly soluble ingredient is excellent only when the amount of the sodium dilauramidoglutamide lysine is more than 0.6 parts by weight relative to 1 part by weight of hydrogenated lecithin.

### Experimental Example 2: Comparison of Formulation Appearance and Stability of Cosmetic Compositions Containing Poorly Soluble Ingredient

2-1. Preparation of Cosmetic Compositions Containing Poorly Soluble Ingredient

It can be verified from Experimental Example 1 above that the composition for stabilizing a poorly soluble ingredient of Example 4, in which the mixing ratio of the hydrogenated lecithin and the anionic surfactant was 1:0.2, showed the highest stability. Furthermore, actual cosmetic compositions were prepared by applying a poorly soluble ingredient to the composition for stabilizing a poorly soluble ingredient of Example 4.

Specifically, cosmetic compositions were prepared using the ingredients and contents shown in Table 6 below. First, an oily phase and an aqueous phase were separately dissolved by heating at 65° C. The oily phase was slowly added while the aqueous phase was stirred by using a homomixer at 2000 rpm. After the oily phase was completely added, stirring was conducted using the homomixer at 2000-2500 rpm for 3 minutes, followed by cooling to 28° C. to thereby prepare the cosmetic compositions. The cosmetic compositions containing the corresponding poorly soluble effective ingredient were prepared as skin formulations.

TABLE 6

| | | | | | Comparative | Comparative |
|---|---|---|---|---|---|---|
| Classification | Raw material | Example 9 | Example 10 | Example 11 | Example 1 | Example 2 |
| Oily phase | Ethanol | 2 | 2 | 2 | 2 | 2 |
| | Dipropylene | 1 | 1 | 1 | 1 | 1 |
| | glycol | | | | | |
| | Hydrogenated | — | 0.2 | 0.2 | — | — |
| | polyisobutene | | | | | |
| | Cedrol | 0.05 | 0.05 | 0.1 | 0.05 | 0.1 |
| | Hydrogenated | 1 | 1 | 1 | 1 | 1 |
| | lecithin | | | | | |
| | Cetyl phosphate | 0.2 | 0.2 | 0.2 | — | — |
| | PEG-40 | — | — | — | 0.3 | 0.3 |
| | hydrogenated | | | | | |
| | caster oil | | | | | |
| | Fragrance | 0.1 | 0.1 | 0.1 | 0.1 | 0.1 |
| Aqueous | Purified water | Remainder | Remainder | Remainder | Remainder | Remainder |
| phase | Arginine | 0.1 | 0.1 | 0.1 | — | — |
| | Glycerin | 8 | 8 | 8 | 8 | 8 |
| | Propanediol | 5 | 5 | 5 | 5 | 5 |
| | 1,2-Hexanediol | 1.5 | 1.5 | 1.5 | 1.5. | 1.5 |
| | Panthenol | 1.5 | 1.5 | 1.5 | 1.5 | 1.5 |

2-2. Comparison of Formulation Appearance and Stability

To find the conditions suitable for the poorly soluble effective ingredient (cedrol), the formulation stability of the cosmetic compositions of Examples 9 to 11 and Comparative Examples 1 and 2 was evaluated. For stability evaluation, each formulation was placed in a transparent plastic container, which was then capped and sealed, and stored under the room-temperature condition, followed by evaluation with the naked eye and a microscope. The results are shown in Table 7.

TABLE 7

| | | | | Comparative | Comparative |
|---|---|---|---|---|---|
| | Example 9 | Example 10 | Example 11 | Example 1 | Example 2 |
| Immediately | Transparent | Transparent | Transparent | Transparent | Transparent |
| after preparation | | | | | |
| Next day (room | Appearance | Appearance | Appearance | Appearance | Precipitation |
| temperature) | maintained | maintained | maintained | maintained | |
| One week after | Appearance | Appearance | Appearance | Precipitation | Precipitation |
| preparation (room | maintained | maintained | maintained | | |
| temperature) | | | | | |
| Four weeks after | Precipitation | Appearance | Appearance | Precipitation | Precipitation |
| preparation (room | | maintained | maintained | | |
| temperature) | | | | | |

As shown in Table 7, precipitation did not occur under the room-temperature condition in Examples 9 to 11, employing hydrogenated lecithin and cetyl phosphate. However, precipitation occurred from day 1 after preparation in Comparative Example 2, in which the content of cedrol was increased without the addition of cetyl phosphate.

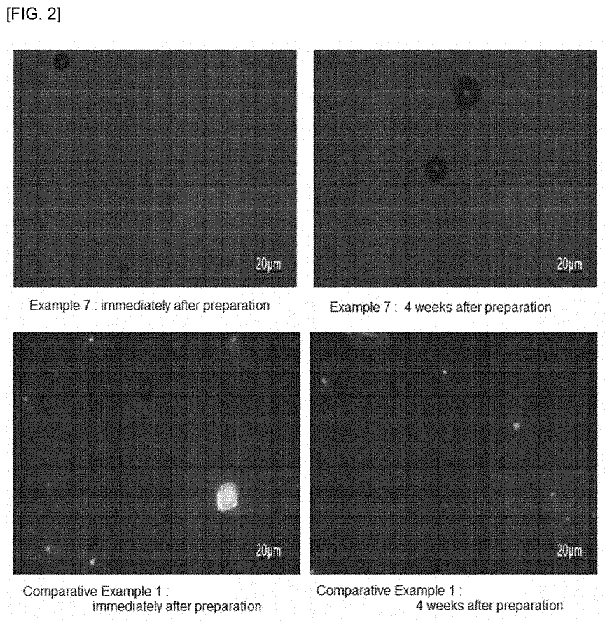

For comparison of long-term stability, precipitation occurred within one week after preparation in both Comparative Examples 1 and 2, corresponding to typical solubilizing formulations without the addition of cetyl phosphate. Meanwhile, precipitation occurred four weeks after preparation in Example 9, without hydrogenated polyisobutene. The appearance was maintained without precipitation even four weeks after preparation in Examples 10 and 11, employing hydrogenated polyisobutene. The formulation stability over time of Example 10 and Comparative Example 1 was observed with a polarization microscope, and the results are shown in FIG. 2.

It was verified through the above results that in the cosmetic compositions containing hydrogenated lecithin and cetyl phosphate, the stability of a poorly soluble ingredient, such as cedrol, was increased, and the application of an oil, such as hydrogenated polyisobutene, together with the above ingredients was more advantageous in long-term stability.

### Experimental Example 3: Comparison of Formulation Appearance and Stability Depending on Viscosity of Cosmetic Composition

3-1. Preparation of Low-Viscosity and High-Viscosity Transparent Cosmetic Compositions

Low-viscosity and high-viscosity transparent cosmetic compositions were prepared using the ingredients and contents shown in Table 8 below. The preparation was performed using the same method as in Experimental Example 2-1 above.

TABLE 8

| Classi- | | Example | Example | Example |
|---|---|---|---|---|
| fication | Raw material | 12 | 13 | 14 |
| Oily phase | Ethanol | 2 | 2 | 2 |
| | Dipropylene glycol | 1 | 1 | 1 |
| | Cedrol | 0.1 | 0.1 | 0.1 |
| | Hydrogenated | 0.2 | 0.2 | 0.2 |
| | polyisobutene | | | |
| | Hydrogenated lecithin | 1 | 4 | 5 |
| | Cetyl phosphate | 0.2 | 0.8 | 1.0 |
| | Fragrance | 0.1 | 0.1 | 0.1 |
| Aqueous | Purified water 1 | Remainder | Remainder | Remainder |
| phase | Arginine | 0.28 | 0.48 | 0.68 |
| | Glycerin | 8 | 8 | 8 |
| | Propanediol | 5 | 5 | 5 |
| | 1,2-Hexanediol | 1.5 | 1.5 | 1.5 |
| | Panthenol | 1.5 | 1.5 | 1.5 |
| Polymer | Purified water 2 | 20 | 20 | 20 |
| phase | Carbomer | 0.18 | 0.18 | 0.18 |

3-2. Comparison of Formulation Appearance and Stability

To investigate the stability of various formulations (low-viscosity and high-viscosity transparent formulations) containing a poorly soluble effective ingredient, the stability of Examples 12 to 14 was evaluated. For stability evaluation, each formulation was placed in a transparent plastic container, which was then capped and sealed, and stored under the conditions of room temperature and −20° C., followed by evaluation with the naked eye. The results are shown in Table 9.

TABLE 9

| | Example 12 | Example 13 | Example 14 |
|---|---|---|---|
| Immediately after | Transparent low- | Transparent | Transparent gel |
| preparation | viscosity skin phase | essence phase | cream phase |
| Next day (room | Appearance | Appearance | Appearance |
| temperature) −20° C. | maintained | maintained | maintained |
| | No precipitation | No precipitation | No precipitation |
| One week after | Appearance | Appearance | Appearance |
| preparation (room | maintained | maintained | maintained |
| temperature) −20° C. | No precipitation | No precipitation | No precipitation |
| Four weeks after | Appearance | Appearance | Appearance |
| preparation (room | maintained | maintained | maintained |
| temperature) −20° C. | No precipitation | No precipitation | No precipitation |

It was verified as shown in Table 9 above that the appearance was stably maintained without precipitation of cedrol, up to four weeks after preparation, in all of Examples 12 to 14 as various types of formulations. That is, it can be seen that the cosmetic compositions prepared in the present invention can stabilize a poorly soluble ingredient in various types of formulations.

While the present invention has been described with reference to the particular illustrative embodiments, a person skilled in the art to which the present invention pertains will be able to understand that the present invention may be embodied in other specific forms without departing from the technical spirit or essential characteristics thereof. Therefore, the examples described above should be construed as exemplifying and not limiting the present invention. The scope of the present invention is not defined by the detailed description as set forth above but by the accompanying claims of the invention, and it should also be understood that all changes or modifications derived from the definitions and scopes of the claims and their equivalents fall within the scope of the invention.

1. A method for stabilizing a poorly soluble ingredient in a cosmetic composition, the method comprising:
mixing hydrogenated lecithin and an anionic surfactant with the poorly soluble ingredient.

2. The method of claim 1, wherein the anionic surfactant is cetyl phosphate, sodium dilauramidoglutamide lysine, or a mixture thereof.

3. The method of claim 1, wherein a mixing ratio of the hydrogenated lecithin and the anionic surfactant is 1:0.005-40 on a weight basis.

4. The method of claim 1, wherein the hydrogenated lecithin is contained in a content of 0.1 wt % to 20 wt % relative to a total weight of the cosmetic composition.

5. The method of claim 1, wherein the anionic surfactant is contained in a content of 0.001 wt % to 4 wt % relative to a total weight of the cosmetic composition.

6. The method of claim 1, said cosmetic composition further comprising an oil.

7. The method of claim 1, wherein the cosmetic composition comprises a micelle containing the poorly soluble ingredient, the hydrogenated lecithin and the anionic surfactant.

8. The method of claim 1, wherein the poorly soluble ingredient is at least one selected from the group consisting of cedrol, formononetin, magonolol, honokiol, phloretin, ceramide, a titrated extract of Centella asiatica, fisetin, daidzein, genistein, glycitein, adenosine, polydatin, retinol, γ-aminobutyric acid, arbutin, macelignan, acetyl phytosphingosine, hydroquinone, hydroxyanisole, ascorbic acid, kojic acid, and retinoids.

9-12. (canceled)
